(19) United States
(12) Patent Application Publication
(10) Pub. No.: US 2008/0242988 A1
(43) Pub. Date: Oct. 2, 2008

(54) ULTRASOUND DIAGNOSTIC APPARATUS, ULTRASOUND IMAGE PROCESSING APPARATUS, AND ULTRASOUND IMAGE PROCESSING METHOD

(76) Inventors: Tetsuya YOSHIDA, Nasushiobara-shi (JP); Naohisa Kamiyama, Otawara-shi (JP); Akihiro Kakee, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/056,635

(22) Filed: Mar. 27, 2008

(30) Foreign Application Priority Data
Mar. 28, 2007 (JP) 2007-085917

(51) Int. Cl. A61B 8/00

(52) U.S. Cl. 600/443

(57) ABSTRACT

There is provided an automatic adjustment function of the dynamic range that carries out statistic processing by the use of each brightness of image data that corresponds to a plurality of frames, automatically determines the upper limit of the dynamic range based on the brightness that corresponds to an echo signal from a subject, and automatically determines a lower limit value of the dynamic range based on the brightness that corresponds to a noise level. By this function, in the case where the dynamic range first established by manual operation, etc. is excessively wide or excessively narrow, the dynamic range can be automatically adjusted to an optimal setting even when the brightness distribution on the image is dynamically varied.

### CROSS-REFERENCE TO RELATED APPLICATIONS

This application is based upon and claims the benefit of priority from prior Japanese Patent Application No. 2007-085917, filed Mar. 28, 2007, the entire contents of which are incorporated herein by reference.

### BACKGROUND OF THE INVENTION

1. Field of the Invention

The present invention relates to an ultrasound diagnostic apparatus, ultrasound image processing apparatus, and ultrasound image processing method which can automatically adjust settings of GAIN and dynamic range that are difficult to predict in advance in a contrast echo method using an ultrasound contrast medium to thereby achieve better visibility.

2. Description of the Related Art

An ultrasound diagnostic apparatus is a diagnostic apparatus that displays images of in vivo information and is utilized as a useful apparatus that is inexpensive and free of radiation exposure and achieves noninvasive and real-time observation, as compared to X-ray diagnostic apparatus, computerized transverse axial tomography, and other image diagnostic apparatus. Because of these characteristics, an ultrasound diagnostic apparatus is capable of wide application, and is used for diagnosis of the heart and other circulatory organs, abdominal area such as the liver, kidney, etc., peripheral blood vessels, Department of Obstetrics and Gynecology, cerebral blood vessels.

In recent years, ultrasound contrast media that can be intravenously administered have been commercialized and a contrast echo method is begun to be practiced. This technique intends to amplify blood-flow signals by injecting ultrasound contrast media intravenously, for example, in tests of the heart, liver, etc. and to assess a blood flow dynamic state. Many of the contrast media adopt microbubbles that function as reflection sources. Ultrasound diagnostic apparatus manufacturers exercise their ingenuity in a reception and transmission method in order to image reflection signals from contrast media highly sensitively, and thus nonlinear signal components from bubbles can be received at high sensitivity.

Now, in an ultrasound diagnostic apparatus, there are so many parameters that must be adjusted when diagnostic imaging is performed, and the operation, therefore, can be said to be complicated. With respect to this problem, each manufacturer has developed a user support function and addresses the problem by mounting automatic adjustment functions, etc. for various gains and STC (Sensitivity Time Control) on their apparatuses.

There still exist, however, following problems in the case where pictures are taken by a contrast echo method by the use of a conventional ultrasound diagnostic apparatus.

That is, the signal brightness level obtained when a contrast medium is administered is frequently unknown until the contrast medium is actually administered. Even when the same liver is observed, the brightness varies in accordance with patients, frequency, and other parameters or by how the probe is applied. That is, the dynamic range randomly set before a contrast medium is administered may be too wide or too narrow, and the brightness may be saturated.

The contrast medium echo method has characteristics in that an image is varied from dark, then, bright, and then, dark as the contrast medium flows in (as time passes). For example, when the blood flow of the liver is observed by a contrast medium, at first, the artery is dyed swiftly, and then after an interval, the portal vein is dyed. In this way, the brightness indicates not a simple change such as monotonic increase but a complicated time change. Consequently, even after a contrast medium is administered, there are cases in which the dynamic range adjusted to a certain timing becomes inappropriate in other timing. In addition, the dynamic range adjustment method would vary in accordance with the objectives as to whether the user wants to see the blood current that flows in blood vessels or perfusion.

### BRIEF SUMMARY OF THE INVENTION

The present invention has been made in view of the above-mentioned circumstances, and it is an object of the present invention to provide an ultrasound diagnostic apparatus or ultrasound image processing apparatus, and a control program therefor, which can automatically and optimally adjust the dynamic range that is difficult to predict in advance in ultrasound imaging.

According to an aspect of the present invention, there is provided an ultrasound diagnostic apparatus comprising: an acquisition unit which acquires a plurality of first image data that correspond to a plurality of frames; a determination unit which determines an upper limit value in accordance with brightness that corresponds to an echo signal from a subject, of said plurality of first image data; and an image generation unit which generates second image data that corresponds to at least one of said plurality of frames by the use of said plurality of image data and a dynamic range defined by at least the upper limit value.

According to another aspect of the present invention, there is provided an ultrasound image processing apparatus comprising: a storage unit which stores a plurality of first image data that correspond to a plurality of frames; a determination unit which determines a upper limit value based on brightness that corresponds to an echo signal from a subject, of said plurality of first image data; and an image generation unit which generates second image data that corresponds to at least one of said plurality of frames by the use of said plurality of image data and a dynamic range defined by at least the upper limit value.

According to yet another aspect of the present invention, there is provided an ultrasound image processing method comprising: deciding an upper limit value based on brightness that corresponds to an echo signal from a subject of a plurality of first image data that correspond to a plurality of frames; and generating second image data that corresponds to at least one of said plurality of frames by the use of said plurality of image data and a dynamic range defined by at least the upper limit value.

### BRIEF DESCRIPTION OF THE SEVERAL VIEWS OF THE DRAWING

FIG. 1 is a block diagram showing an ultrasound diagnostic apparatus 10 according to an embodiment;

FIG. 2 is a view showing dedicated buttons, etc. to input ON/OFF instructions of an automatic adjustment function of a dynamic range;

FIG. 3 is a flow chart showing a flow of automatic adjustment processing of a dynamic range;

FIG. 4 is a view showing one example of a histogram concerning brightness that follows a first dynamic range;

FIG. 5 is a view for explaining a second dynamic range adjusted by the automatic adjustment processing of the dynamic range;

FIG. 6 is a view showing one example of an ultrasound image obtained in conformity to the first dynamic range;

FIG. 7 is a view showing one example of an ultrasound image obtained in conformity to the second dynamic range adjusted by the automatic adjustment processing of the dynamic range;

FIG. 8 is a view showing another example of an ultrasound image obtained in conformity to the first dynamic range;

FIG. 9 is a view showing maximum brightness (tone) Xmax and minimum brightness (tone) Xmin (that is, time changes of maximum brightness Xmax and minimum brightness Xmin) in each frame;

FIG. 10 is a view illustrating the effect when the automatic adjustment processing of the dynamic range is applied to an ultrasound dynamic image whose brightness changes with time;

FIG. 11 is a view illustrating the effect when the automatic adjustment processing of the dynamic range is applied to an ultrasound dynamic image whose brightness changes with time;

FIG. 12 is a view illustrating the effect when the automatic adjustment processing of the dynamic range is applied to an ultrasound dynamic image whose brightness changes with time;

FIG. 13 is a view illustrating the effect when the automatic adjustment processing of the dynamic range is applied to an ultrasound dynamic image whose brightness changes with time;

FIG. 14 is a view illustrating the effect when the automatic adjustment processing of the dynamic range is applied to an ultrasound dynamic image whose brightness changes with time;

FIG. 15 is a flow chart showing a flow of processing when the automatic adjustment function of the dynamic range is applied in the case where a dynamic study (dynamic observation of blood flow) is conducted;

FIG. 16 is a flow chart showing a flow of processing when the automatic adjustment function of the dynamic range is applied in the case where reperfusion is observed; and

FIG. 17 is a flow chart showing a flow of processing (fine adjustment processing of the dynamic range) that conforms to a fine adjustment function of the dynamic range according to the second embodiment.

### DETAILED DESCRIPTION OF THE INVENTION

Referring now to the accompanying drawings, embodiments according to the present invention will be described. Incidentally, in the following description, constituent elements having substantially the same functions and configurations will be assigned the same reference numerals and signs, and they will be repeatedly described only on necessary occasions.

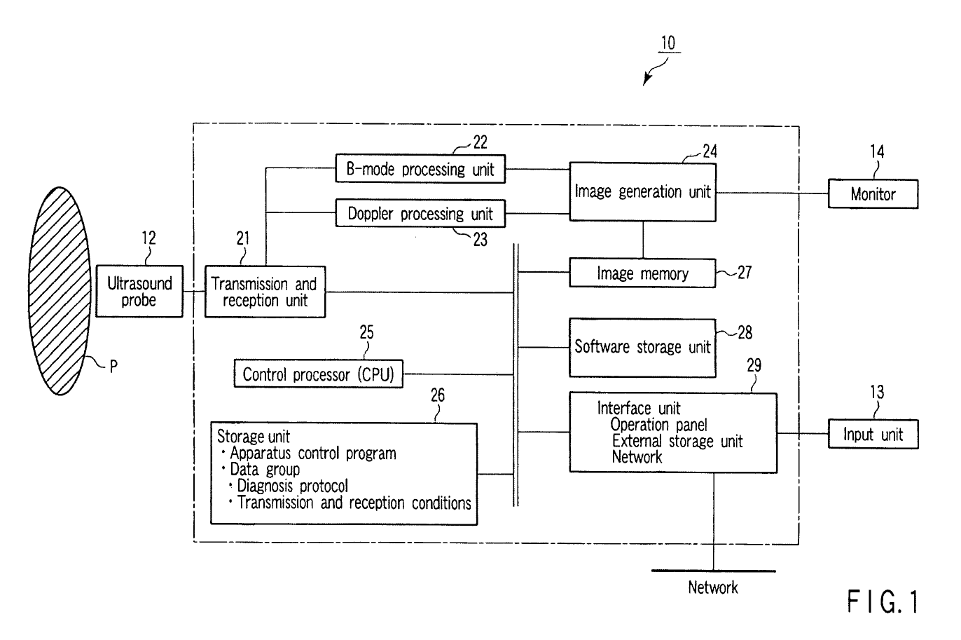

FIG. 1 is a block diagram showing an ultrasound diagnostic apparatus 10 according to this embodiment. As shown in the figure, the ultrasound diagnostic apparatus 10 includes a ultrasound probe 12, an input unit 13, a monitor 14, a transmission and reception unit 21, a B-mode processing unit 22, a Doppler processing unit 23, an image generation unit 24, a control processor (CPU) 25, a memory unit 26, an image memory 27, a software storage unit 28, and an interface unit 29.

The ultrasound probe 12 has piezoelectric vibrators as acoustoelectric reversible transducer elements such as piezoelectric ceramics. A plurality of piezoelectric vibrators are arrayed in parallel and equipped to the leading end of the probe 12.

The ultrasound probe 12 may be able to ultrasonically scan a three-dimensional region of a subject. In such event, the ultrasound probe 12 has a construction to mechanically oscillate vibrators along the direction orthogonal to their arrangement direction and ultrasonically scan the three-dimensional region, or a construction to ultrasonically scan the three-dimensional region by electrical control by the use of two-dimensional oscillating elements arranged two-dimensionally. In the case where the former construction is adopted, three-dimensional scan of a subject is conducted by the oscillation circuit, and a testing person is able to obtain automatically a plurality of two-dimensional tomograms only by bringing the probe proper in contact with the subject. From the controlled oscillation speed, the accurate distance between cross sections can be detected. In addition, in the case where the latter configuration is adopted, in principle, the three-dimensional region can be ultrasonically scanned in the same time required for obtaining conventional two-dimensional tomograms.

The input unit 13 has switches, buttons, a mouse, a keyboard, and a track ball for inputting various instructions, directions, and information.

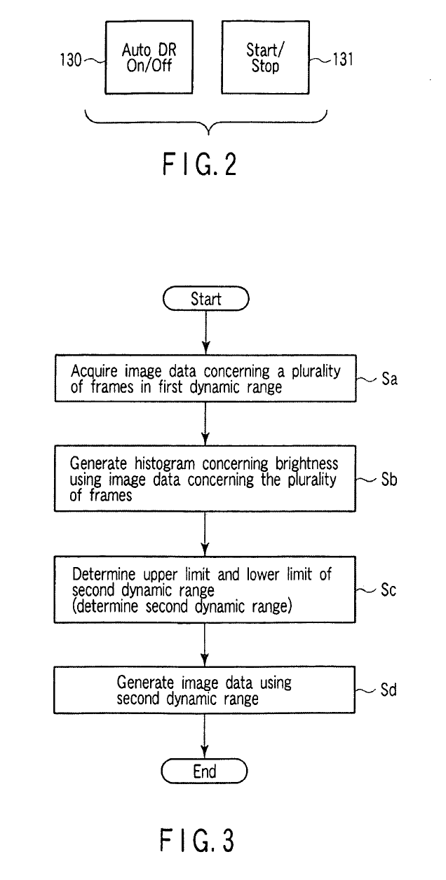

Incidentally, the input unit 13 has an ON/OFF button 130 to give ON/OFF instructions of an operation mode of the automatic adjustment function of the dynamic range later discussed, and a START/STOP button 131 to designate a target period to which the automatic adjustment function of the dynamic range is applied, as shown in FIG. 2.

The monitor 14 displays morphological information (B-mode image), blood flow information (average speed image, dispersion image, power image, etc.), ultrasound images that conform to the dynamic range optimized by the dynamic range adjustment function later discussed, and the like within the living body in predetermined forms in accordance with video signals from the image generation unit 24.

The transmission and reception unit 21 has a trigger generating circuit, a delay circuit, a pulser circuit, etc., which are not illustrated. In the pulser circuit, the rate pulse for forming transmission ultrasound waves is repeatedly generated at a predetermined rate frequency fr Hz (cycle: 1/fr seconds). In addition, in the delay circuit, the delay time necessary for focusing ultrasound waves into a beam form for each channel and for determining transmission directivity is given to each rate pulse. By varying this delay information, the transmission direction from the probe vibrator surface can be arbitrarily adjusted. The trigger generation circuit applies a drive pulse to the probe 12 at a timing based on this rate pulse.

The transmission and reception unit 21 has a function to instantaneously change delay information, transmission frequency, transmission drive voltage, etc. in accordance with the instructions of the control processor 25. In particular, the transmission drive voltage is changed by a linear amplifier type transmission circuit that can instantaneously change over the voltage value or a mechanism that electrically changes over a plurality of power supply units.

The transmission and reception unit 21 includes an amplifier circuit, an A/D converter, an adder, etc., which are not illustrated. In the amplifier circuit, echo signals imported via the probe 12 are amplified for each channel. In the A/D converter, delay time necessary to determine the reception directivity is given to the amplified echo signals, and thereafter, addition processing is performed in the adder. By this addition, a reflection component from the direction that corresponds to the reception directivity of the echo signals is emphasized and, by the reception directivity and the transmission directivity, a comprehensive beam of ultrasound transmission and reception is formed.

The B-mode processing unit 22 receives echo signals from the transmission and reception unit 21, executes logarithmic amplification, envelop wave detection processing, and the like, and generates data in which the signal intensity is expressed by the degree of brightness.

The Doppler processing unit 23 frequency-analyzes speed information from the echo signals received from the transmission and reception unit 21, extracts blood flow, tissue, and contrast medium echo components by Doppler effects, and finds average speed, dispersion, power, and other blood flow information at multipoints.

The image generation unit 24 converts scanning line signal columns of ultrasound scan into scanning line signal columns of a general video format represented by TV, etc. and generates ultrasound diagnostic images to be displayed. Note that, the data before it enters the relevant image generation unit 23 is sometimes called “raw data.”

The control processor 25 has a function as an information processor (computer) and controls the action of the whole ultrasound diagnostic apparatus. The control processor 25 reads a dedicated program to achieve the automatic adjustment function of the dynamic range, a predetermined scan sequence, a control program for executing image generation, display, etc. from the storage unit 26, deploys them on the software storage unit 28, and executes the operation, control, etc. concerning various kinds of processing.

The storage unit 26 is a recording medium such as a magnetic disk (floppy (registered trademark) disk, hard-disk, etc.), optical disk (CD-ROM, DVD, etc.), and semiconductor memory and is a device to read information recorded in these media. This storage unit 26 stores various scan sequences, a dedicated program for achieving the automatic adjustment function of the dynamic range later discussed, a control program for executing image generation and display processing, diagnostic information (patient IDs, remarks of physicians, etc.), diagnostic protocol, transmission and reception conditions, and other data groups. In addition, the storage unit 26 is used for storage of images of the image memory 27 as required. The data in the storage unit 26 can be transferred to external peripheral devices via the interface unit 29.

The image memory 27 stores image data generated at the image generation unit 24. The image data stored in this image memory 27 has a dynamic range about twice as much as the image data displayed on the monitor 14. The image data stored in the image memory 27 can be fetched by the operator after diagnosis and can be reproduced in a still image or in a moving image using a plurality of image data. In addition, the image memory 27 stores output signals right after the ultrasound transmission and reception unit 21 (called radio frequency (RF) signal), image brightness signal right after the signal passes the transmission and reception unit 21, other raw data, image data obtained via a network, and the like as required.

The interface unit 29 is an interface concerning the input unit 13, network, and a new external storage unit (not illustrated). The data of ultrasound images and analysis results, etc. obtained by the relevant apparatus can be transferred to other apparatus via a network by the interface unit 29.

(Automatic Adjustment Function of the Dynamic Range)

Next discussion will be made on the automatic adjustment function of a dynamic range which this ultrasound diagnostic apparatus 10 possesses. This function automatically adjusts the dynamic range, for example, in cases where brightness of an image fluctuates with time, such as in the contrast medium echo method.

Incidentally, in this embodiment, the description will be made on an example in which the automatic adjustment function of the dynamic range is achieved by the ultrasound diagnostic apparatus 10. The embodiment is not limited to this example, and it may be achieved by an ultrasound image processing apparatus such as a workstation to which a program that achieves the automatic adjustment function of the dynamic range is installed.

FIG. 3 is a flow chart showing a flow of processing (dynamic range automatic adjustment processing) that conforms to the automatic adjustment function of the dynamic range.

In the figure, first of all, the control processor 25 scans a subject with an contrast medium injected by ultrasound and a plurality of first image data that correspond to a plurality of frames are obtained by the use of a first dynamic range (Step Sa).

In this event, the first dynamic range is to be established in the preceding stage of automatic adjustment processing of the dynamic range by initial setting, manual setting, etc. In addition, in the scan sequence in this step, there is no limitation as long as the scan sequence conforms to the contrast medium echo method.

Next, when the control processor 25 receives an instruction to initiate the automatic adjustment processing via the input unit 13, the control processor 25 generates a histogram concerning brightness using the first image data that corresponds to the plurality of frames acquired in Step S1 (Step Sb).

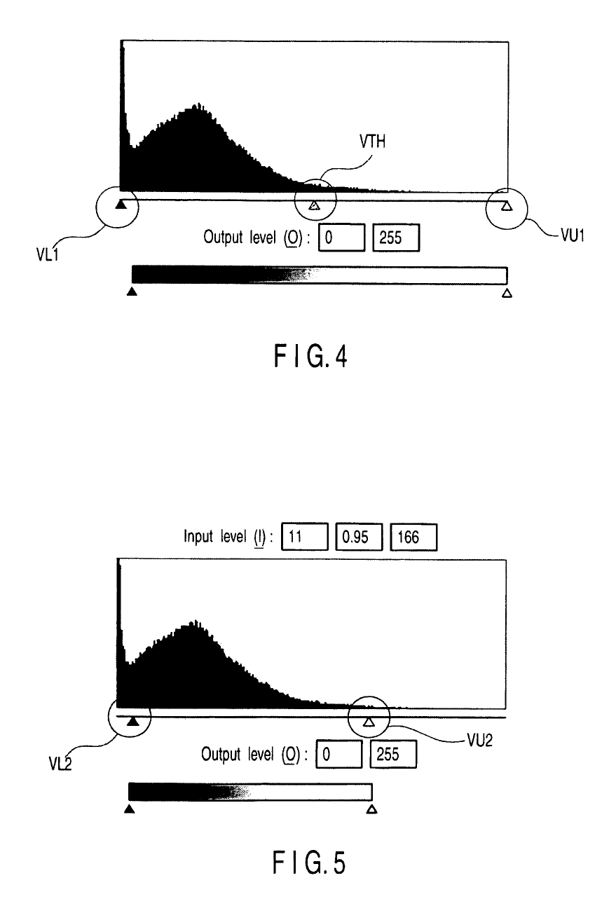

FIG. 4 shows one example of histograms generated in Step S2. Note that, in the figure, the upper limit of the first dynamic range is shown by V_{U1} and the lower limit thereof by V_{L1}, respectively.

Then, the image generation unit 24 determines the upper limit V_{U2} and the lower limit V_{L2} of the second dynamic range by the use of the generated histogram concerning brightness (Step Sc). That is, the image generation unit 24 determines the maximum value of brightness exceeding the predetermined threshold value V_{TH} in the histogram generated in Step S2 as the upper limit V_{U2} of the second dynamic range. In addition, the image generation unit 24 determines the brightness that corresponds to the white noise level in the histogram as the lower limit V_{L2} of the second dynamic range. Incidentally, in FIG. 5, the upper limit V_{U2} and the lower limit V_{L2} of the second dynamic range set to the histogram are shown.

The method of determining the upper limit V_{U2} of the second dynamic range is not limited to the above example. For example, a value which is lower than the predetermined maximum value by a predetermined rate (for example, 10%) of the range from the lower limit V_{L2} to the predetermined maximum value may be determined as the upper limit V_{U2} of the second dynamic range. Further, a histogram concerning the brightness value may be generated for each image in the Step Sb, the maximum brightness value for each frame may be calculated in Step Sc, and the whole maximum value may be calculated by using these values. Furthermore, an image having the maximum brightness value of a plurality of images included in the target period T may be selected, and the upper limit V_{U2} of the second dynamic range may be determined by using the image.

In order to make the determination of the lower limit V_{L2} of the second dynamic range more suitable, it is preferable that at least one of the plurality of frames that correspond to the image data obtained in Step S1 be a frame in which ultrasound reception after priming has been executed (ultrasound reception only has been executed without conducting ultrasound transmission). In particular, it is preferable that it be a frame that corresponds to right after a contrast medium is administered, a frame that corresponds to after a predetermined period passes after a contrast medium is administered (for example, 10 seconds after the contrast medium administration start button is operated), a frame that corresponds to right after high sound pressure is transmitted, etc. This enables brightness that corresponds to the white noise level to be accurately included in the histogram generated in Step S2.

Next, the image generation unit 24 acquires a plurality of second image data that correspond to the same plurality of frames, of the first image data generated in Step S1, by the use of signals having brightness that belongs to the second dynamic range (Step Sd).

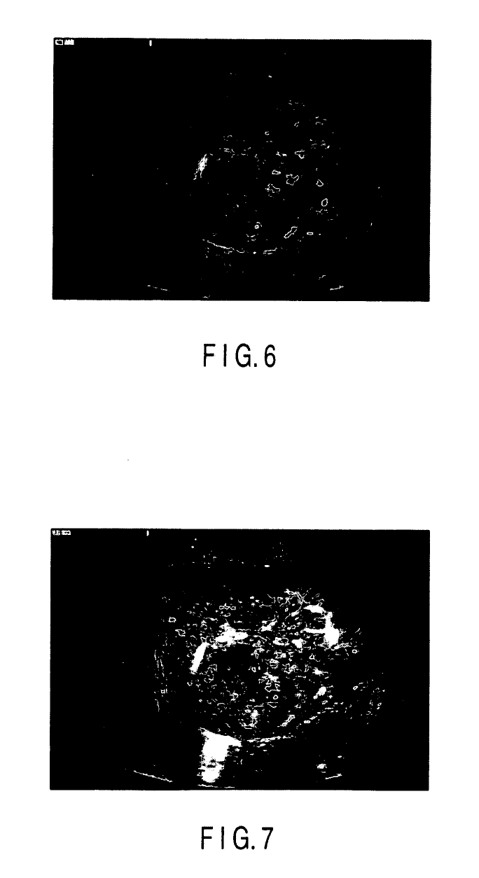

As described above, by the automatic adjustment processing of the dynamic range, the first dynamic range established by a regular technique (see FIG. 4) is automatically adjusted to a more preferable second dynamic range (see FIG. 5) for the contrast medium echo method. Consequently, even when a displayed image becomes dark as shown in FIG. 6 because the first dynamic range established by a regular technique is too wide, it is possible to establish a tone suited for observation as shown, for example, in FIG. 7 by optimizing the dynamic range by this automatic adjustment function.

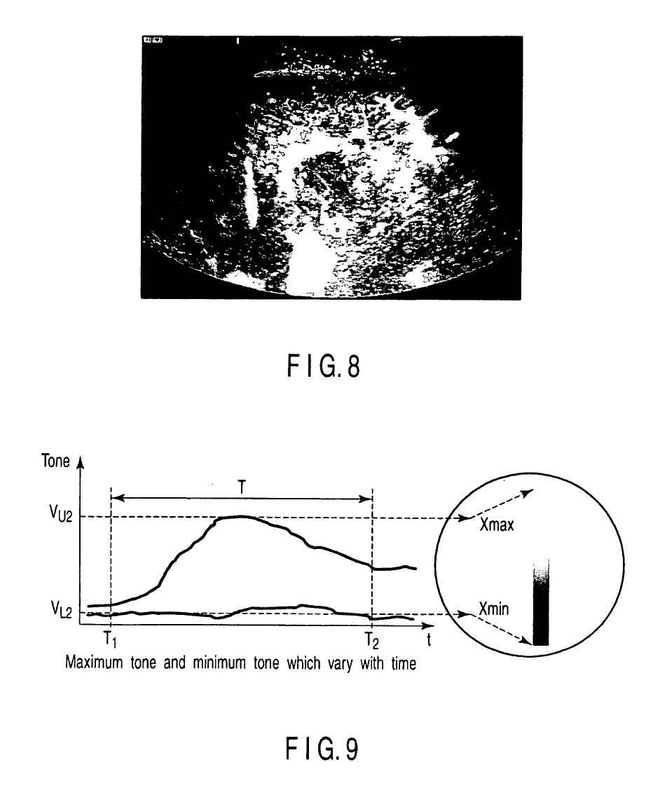

Incidentally, in the above description, too wide a first dynamic range is changed to a narrower second dynamic range by the already described automatic adjustment function. Irrespective of the relevant example, this automatic adjustment function can be applied to a case in which, for example, an excessively narrow first dynamic range is changed to a wider second dynamic range by this automatic adjustment function. In such event, too, an image in which a signal is almost saturated as shown, for example, in FIG. 8 because the first dynamic range is too narrow can be changed to have a tone suited for observation as shown in FIG. 7.

The automatic adjustment function of the dynamic range can also target moving images (e.g. AVI file, Raw data or the like)

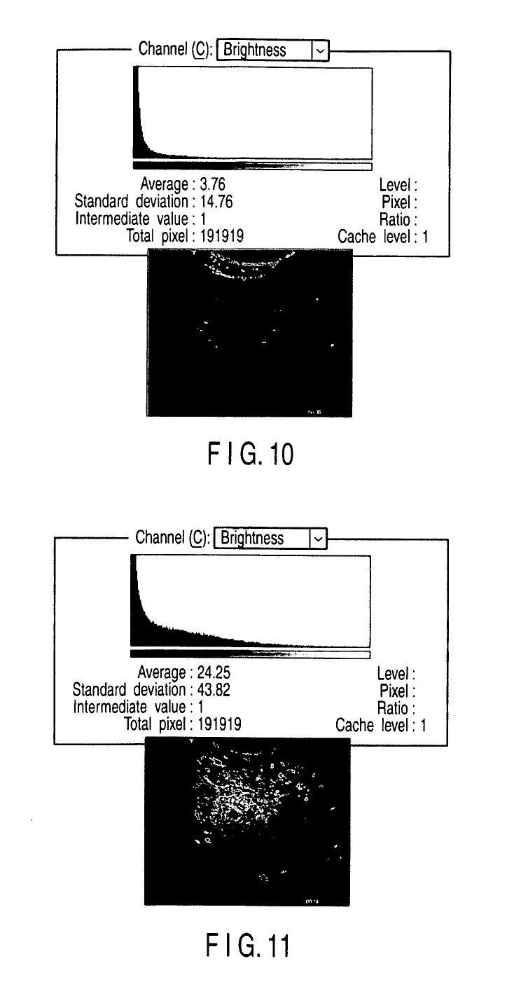

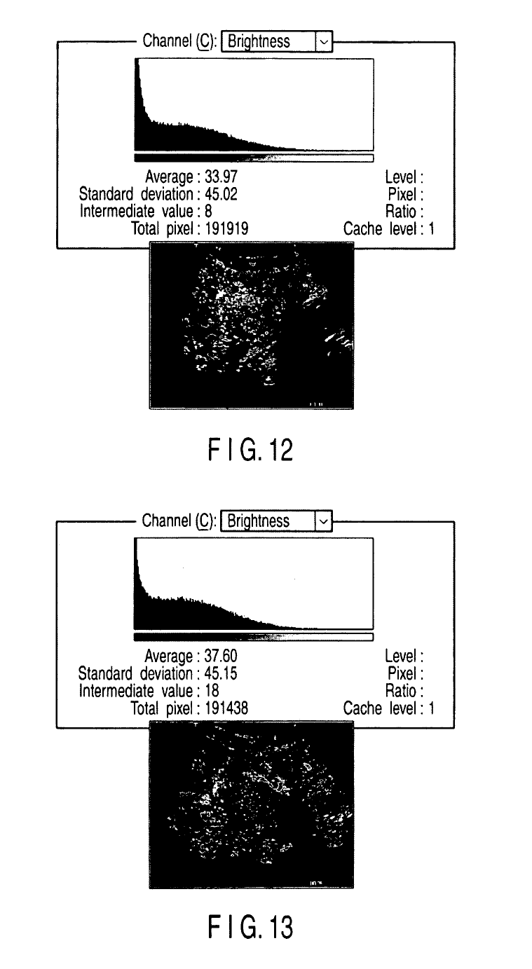

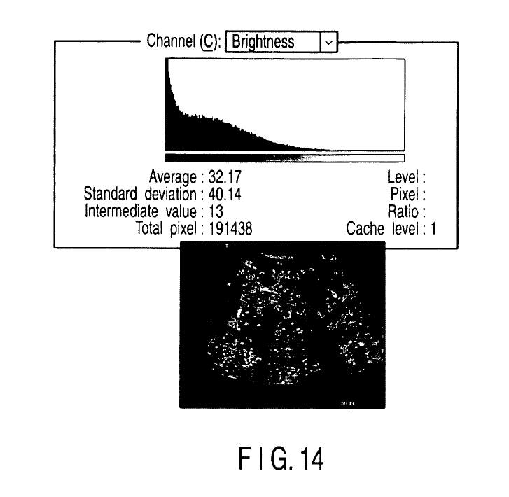

FIG. 9 is a view showing the maximum brightness (tone) Xmax and the minimum brightness (tone) Xmin in each frame (that is, time change of maximum brightness Xmax and minimum brightness Xmin). In the case where this automatic adjustment function is applied to a plurality of frames which belong to a target period T, an ultrasound image that corresponds to each frame in the target period T is generated for the brightness in the second dynamic range with the upper limit set to V_{U2} and the lower limit set to V_{L2}. Consequently, even in the case where the brightness greatly changes with time as shown in histograms of FIGS. 10 to 14, ultrasound moving images whose dynamic range is optimized can be generated and displayed as shown in ultrasound images of FIGS. 10 to 14.

In general, in the contrast medium echo method, it takes time for a contrast medium to flow into an imaging object. In addition, there are cases in which flash transmission and monitoring transmission are repeated and how a contrast medium is reperfused in an imaging object is observed with time. Consequently, in the case where this automatic adjustment function of the dynamic range is applied to moving images which are useful for diagnostic imaging, it is important to suitably determine the start time T1 and the finish time T2 of the target period T.

Consequently, in the ultrasound diagnostic apparatus 10, the target period T, start time T1, and finish time T2 can be determined, for example, by each of the following techniques.

### EXAMPLE 1

The start time T1 and the finish time T2 of the target period T can be determined in accordance with timing in which the start/stop switch 131 shown in FIG. 2 is manually operated. That is, the control processor 25 sets the timing when the start/stop switch 131 is first pressed as the start time T1 and the timing when the start/stop switch 131 is pressed for the second time as the finish time T2 under the automatic adjustment mode of the dynamic range. For the image data that corresponded to a frame in the target period T defined by this T1 to T2, the automatic adjustment processing of the dynamic range is executed.

### EXAMPLE 2

The finish time T2 of the target period T can be determined in accordance with timing of importing action (freeze button operation, etc.) of ultrasound images currently on-camera. That is, when the freeze button is pressed under the automatic adjustment mode of the dynamic range, the control processor 25 sets the time that corresponds to the frame subject to the freeze as the finish time T2, in linkage with the action of the freeze button being pressed. In such event, the start time T1 may be established by any technique.

### EXAMPLE 3

The start time T1 of the target period T can be determined by using the timer ON action timing in starting to inject a contrast medium as a reference. That is, the control processor 25 determines the time after a predetermined period of ON time of the timer as the start time T1, in linkage with the relevant timer ON timing at the start of contrast medium injection. In such event, the finish time T2 may be established by any technique. This technique is preferable, for example, when changes of brightness with time due to a contrast medium are observed.

### EXAMPLE 4

At least one of the start time T1 and finish time T2 of the target period T can be determined with the flash transmission timing used as a reference. That is, the control processor 25 determines the start time T1 in linkage with, for example, the n-th flash transmission (n is a natural number). In addition, the control processor 25 determines the start time T1 in linkage with, for example, the n+1-th flash transmission. This technique is preferable, for example, when the condition of contrast medium reperfusion is observed.

### EXAMPLE 5

The start time T1 and the finish time T2 can be determined posteriori for temporally continuous image data. That is, the control processor 25 cuts out a frame that belongs to the start time T1 to the finish time T2 established by a given technique for temporally continuous image data acquired in advance, and executes the automatic adjustment processing of the dynamic range for the frame.

(Action)

Next, a typical scan sequence including automatic adjustment processing of the dynamic range will be described.

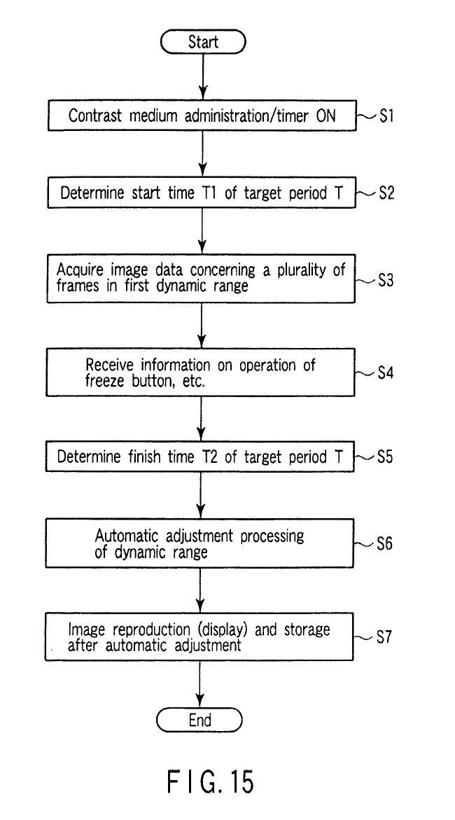

FIG. 15 is a flow chart showing a flow of processing when the automatic adjustment function of the dynamic range is applied in the case where a dynamic study (dynamic observation of blood flow) is conducted. In the figure, first, when a contrast medium starts to be administered, the control processor 25 operates in linkage with this to turn ON a timer for grasping the time passage (Step S1) and determines the time a predetermined time after the timer is turned ON as the start time T1 (Step S2).

Next, the control processor 25 scans a subject with a contrast medium injected by ultrasound wave, and acquires a plurality of first image data that correspond to a plurality of frames by the use of the first dynamic range (Step S3). In such event, the user is able to observe changes of a stained image of an interested region (for example, suspected tumor) with time by the first dynamic range for a predetermined time (for example, about 60 seconds to see wash-in).

Next, when the control processor 25 receives information on the freeze button being operated in desired timing from a user (Step S4), the control processor 25 operates in linkage with the relevant freeze button operation and determines the finish time T2 of the target period T (Step S5). By the determination of the finish time T2 and the start time T1 in Step S2, the second dynamic range is defined. Note that, even after the operation of the freeze button, the scan may be executed to observe a contrast medium remaining in subject organs as required.

Next, the control processor 25 executes the automatic adjustment processing of the dynamic range already discussed and acquires the image data on which brightness is converted in accordance with the second dynamic range (Step S6), automatically loop-reproduces each image, and then, stores moving image data or still image data in the storage unit 26 in conformity to user instructions (Step S7).

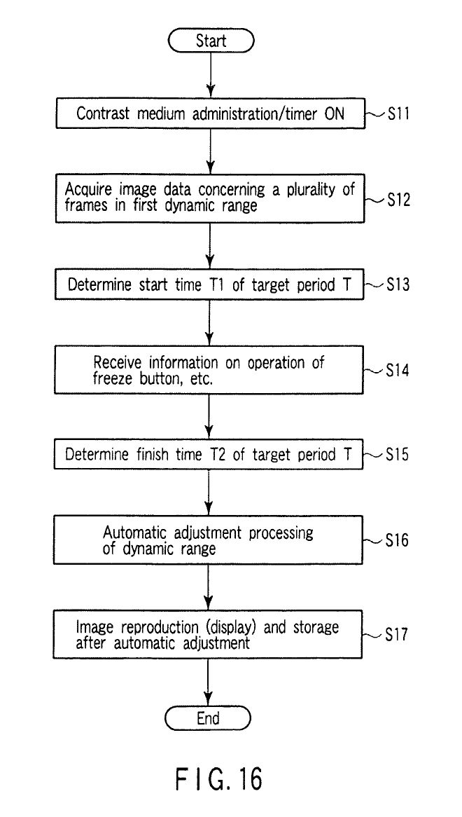

FIG. 16 is a flow chart showing a flow of processing when the automatic adjustment function of the dynamic range is applied in the case where reperfusion is observed. In the figure, first, when a contrast medium starts to be administered, the control processor 25 operates in linkage with this and turns ON the timer to grasp the time passage (Step S11).

Next, in order to observe the reperfusion by the use of a contrast medium, the control processor 25 scans a subject with the contrast medium injected by ultrasound wave in accordance with a scan sequence that includes monitoring transmission (transmission to continuously transmit ultrasound wave of sound pressure that does not destroy contrast medium bubbles and monitor the contrast medium flow-in condition) and flash transmission (transmission to transmit ultrasound wave of sound pressure that can destroy contrast medium bubbles and temporarily reset the contrast medium flow-in from the scan surface), and acquires a plurality of first image data that correspond to a plurality of frames by the use of the first dynamic range (step S12). In addition, the control processor 25 determines the start time T1 in linkage with the flash transmission (Step S13).

Next, when the control processor 25 receives information on the stop button 131 being operated after the brightness reaches a plateau (Step S14), the control processor 25 operates in linkage with the relevant stop button operation and determines the finish time T2 of the target period T (Step S15). By the determination of the finish time T2 and the start time T1 in Step S13, the second dynamic range is defined.

Next, the control processor 25 executes the automatic adjustment processing of the dynamic range already discussed and acquires the image data in accordance with the second dynamic range (Step S16), automatically loop-reproduces each image, and then, stores moving image data or still image data in the storage unit 26 in conformity to user instructions (Step S17).

Incidentally, processing may be executed by combining the sequence shown in FIG. 15 with the sequence shown in FIG. 16 by one administration of a contrast medium.

(Effect)

According to the above-mentioned configuration, the following effects can be achieved.

The present ultrasound diagnostic apparatus uses each brightness of image data that correspond to a plurality of frames to carry out statistical processing, automatically determines the upper limit of the dynamic range based on the brightness that corresponds to an echo signal from a subject, and has an automatic adjustment function of a dynamic range that automatically determines the lower limit of the dynamic range based on the brightness that corresponds to a noise level. Consequently, in the contrast medium echo method in which the signal brightness level is difficult to be determined, even in the case where the dynamic range first established by manual operation, etc. is excessively wide or excessively narrow, the dynamic range can be automatically adjusted to the optimum setting by the use of this function.

In addition, in particular, in the contrast medium echo method, images are varied from dark, bright, and then dark as a contrast medium flows in. Even in the case where the brightness distribution on the image is dynamically varied, by the use of the automatic adjustment function of the dynamic range, the dynamic range can be dynamically adjusted in compliance with changes of the image.

### Second Embodiment

Next, a second embodiment of the present invention will be explained. Generally, when a dynamic range is set, brightness modulation occurs in the set dynamic range, and the image contrast differs from the image contrast before the dynamic range is set. In the first embodiment, explained is an example in which, in the contrast medium echo method, a second dynamic range is set by the above-explained automatic adjustment function, brightness is modulated, and an image having a suitable contrast is provided.

However, there are cases where distribution of brightness values in the image varies, due to individual difference between patients or observed regions. Further, there are cases where it is effective to perform observation in various dynamic ranges, to achieve some diagnosis purposes. In these cases, it is preferable to perform fine adjustment of the dynamic range set by the automatic adjustment function of the first embodiment.

Therefore, in the second embodiment, explained is an ultrasound diagnostic apparatus which can perform fine adjustment of the dynamic range after the second dynamic range is set, such that the contrast has a desired value.

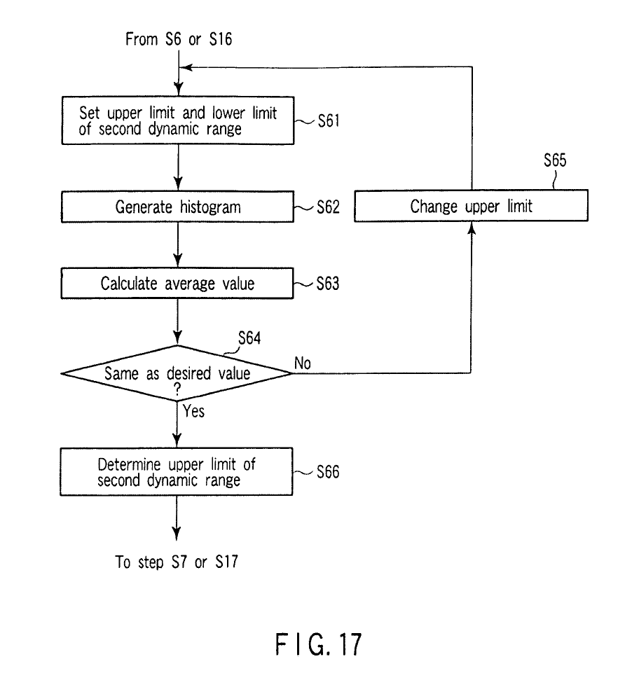

FIG. 17 is a flow chart illustrating a flow of processing (fine adjustment processing of the dynamic range) that conforms to a fine adjustment function of the dynamic range according to the second embodiment. The fine adjustment processing of the dynamic range will be explained below with reference to FIG. 17. The fine adjustment processing of the dynamic range is performed after the dynamic range is set in, for example, Step S6 of FIG. 15 or Step S16 of FIG. 16.

First, the control processor 25 sets the upper limit V_{U2} and the lower limit V_{L2} of the second dynamic range (Step S61), and generates a histogram concerning the brightness by using the limits by the use of all the images included in the target period (Step S62).

Next, the control processor 25 calculates an average value (average brightness value) of the generated histogram (Step S63), and determines whether the obtained average brightness value is the same as a preset desired value or not (or whether the average brightness value falls within a predetermined range set based on the desired value or not) (Step S64).

When it is determined in step S64 that the average brightness value differs from the preset desired value (or that the average brightness value does not fall within the predetermined range set based on the desired value), the control processor 25 changes the upper limit V_{U2} of the second dynamic range (Step S65), and repeats processing of Steps S61 to S64 by the use of a new upper limit V_{U2} of the second dynamic range.

On the other hand, when it is determined that the average brightness value is the same as the preset desired value (or the average brightness value falls within the predetermined range set based on the desired value), the control processor 25 determines the second dynamic range by the use of the current upper limit V_{U2}, and performs processing of Step S7 or Step S17. A setting method of the desired value is not limited. For example, the desired value may be an initial set value (recommended value) of the apparatus, or a value set by manual operation.

A histogram concerning the brightness value may be generated for each image in Step S62, an average brightness value may be calculated for each frame in Step S64, and the whole average value may be calculated by using the values. Further, the fine adjustment processing of the dynamic range can be performed for each frame. In addition, the fine adjustment processing of the dynamic range may be performed only for an image having the maximum brightness value of images included in the target period T.

In the above fine adjustment processing of the dynamic range, explained is the case where adjustment is performed by changing the upper limit value such that the average brightness value matches the desired value. However, the invention is not limited to this example. For example, the upper limit value to match the average brightness value with the desired value may be obtained by predetermined calculation and the like.

According to the above constitution, it is possible to perform fine adjustment of the dynamic range, and set a dynamic range optimum for the user, regardless of individual difference between patients or observed regions. In addition, even when a noise having an extremely high value occurs, it is possible to provide an ultrasound image having a high image quality with stability.

Incidentally, the present invention is not limited to the above-mentioned examples as they are, and in the implementation stages, component elements may be modified and embodied without departing from the spirit and scope of the invention. Specific modified examples include the following.

(1) Each function according to the present embodiment may be achieved by installing programs to execute the relevant processing to workstations and other computers and deploying these on memory. In such event, the programs that allow the computer to execute the relevant techniques may be stored in recording media such as magnetic disks (floppy (registered trademark) disks, hard-disks, etc.), optical disks (CD-ROM, DVD, etc.), semiconductor memory, and others for distribution.

(2) In the case where the automatic adjustment function of the dynamic range according to the present embodiment is achieved by an ultrasound imaging processing apparatus, as the information concerning the lower limit V_{L2} of the dynamic range, not only the lower limit V_{L2} value but also, for example, image data used for deciding the lower limit V_{L2} may be stored in memory, and the lower limit V_{L2} may be determined posteriori by the use of the data. In addition, information on target period T, start time T1, and finish time T2, information on the upper limit V_{U2}, and information on the lower limit V_{L2} are preferably managed and stored in memory as ancillary information of the image subject to the automatic adjustment function of the dynamic range. Furthermore, these pieces of information may be changed posteriori and reestablished as required.

(3) The automatic adjustment function of the dynamic range exhibits effects particularly in the contrast medium echo method, but the application is not limited to this method. For example, even in the case where ultrasound dynamic images are observed without using any contrast medium, suitable ultrasound images can be provided by optimizing the dynamic range by this automatic adjustment function.

Furthermore, by suitably combining a plurality of constituent elements disclosed in the above-mentioned examples, various inventions can be formed. For example, several constituent elements may be deleted from all the constituent elements shown in examples. Furthermore, constituent elements covering different examples may be suitably combined.

What is claimed is:

1. An ultrasound diagnostic apparatus comprising:
an acquisition unit which acquires a plurality of first image data that correspond to a plurality of frames;
a determination unit which determines an upper limit value in accordance with brightness that corresponds to an echo signal from a subject, of said plurality of first image data; and
an image generation unit which generates second image data that corresponds to at least one of said plurality of frames by the use of said plurality of image data and a dynamic range defined by at least the upper limit value.

2. The ultrasound diagnostic apparatus according to claim 1, wherein the determination unit determines a lower limit value based on the brightness that corresponds to a noise level acquired from said plurality of first image data, and
the dynamic range is defined by the upper limit value and the lower limit value.

3. The ultrasound diagnostic apparatus according to claim 1, wherein the determination unit determines the upper limit value based on brightness that corresponds to an echo signal arising from a contrast medium injected into the subject.

4. The ultrasound diagnostic apparatus according to claim 2, wherein the determination unit generates a histogram concerning a tone by the use of said plurality of first image data, and determines at least one of the upper limit value and the lower limit value by the use of the histogram.

5. The ultrasound diagnostic apparatus according to claim 2, wherein at least one of said plurality of frames is a frame on which ultrasound reception only is executed, and
the determination unit determines the lower limit value based on brightness that corresponds to white noise contained in the first image data that corresponds to the frame on which the ultrasound reception only is executed.

6. The ultrasound diagnostic apparatus according to claim 1, further comprising a setting unit which sets at least one of a start time and a finish time on a period of collecting said plurality of first image data.

7. The ultrasound diagnostic apparatus according to claim 6, wherein the setting unit sets the finish time based on an operation timing of a freeze instruction to freeze display of the first image data.

8. The ultrasound diagnostic apparatus according to claim 6, wherein the setting unit sets the start time with timing for injecting a contrast medium into a subject used as a reference.

9. The ultrasound diagnostic apparatus according to claim 6, wherein the setting unit sets the start time with timing of flash transmission used as a reference.

10. The ultrasound diagnostic apparatus according to claim 1, wherein the image generation unit generates the second image data that corresponds to at least one of said plurality of frames in response to a freeze instruction to freeze display of the first image data.

11. The ultrasound diagnostic apparatus according to claim 1, wherein the image generation unit generates a plurality of the second image data using the dynamic range, and
the ultrasound diagnostic apparatus further comprises a reproduction unit which loop-reproduces said plurality of second image data.

12. The ultrasound diagnostic apparatus according to claim 1, wherein the determination unit determines the upper limit value based on maximum brightness of the second image data.

13. An ultrasound image processing apparatus comprising:
a storage unit which stores a plurality of first image data that correspond to a plurality of frames;
a determination unit which determines a upper limit value based on brightness that corresponds to an echo signal from a subject, of said plurality of first image data; and
an image generation unit which generates second image data that corresponds to at least one of said plurality of frames by the use of said plurality of image data and a dynamic range defined by at least the upper limit value.

14. The ultrasound image processing apparatus according to claim 13, wherein the determination unit determines a lower limit value based on brightness that corresponds to a noise level acquired from said plurality of first image data, and
the dynamic range is defined by the use of the upper limit value and the lower limit value.

15. The ultrasound image processing apparatus according to claim 13, wherein the determination unit determines the upper limit value based on brightness that corresponds to an echo signal arising from a contrast medium injected into the subject.

16. The ultrasound image processing apparatus according to claim 14, wherein the determination unit generates a histogram concerning a tone by the use of said plurality of first image data, and determines the upper limit value and the lower limit value by the use of the histogram.

17. The ultrasound image processing apparatus according to claim 14, wherein at least one of said plurality of frames is a frame on which ultrasound reception only is executed, and
the determination unit determines the lower limit value based on brightness that corresponds to white noise contained in the first image data that corresponds to the frame on which the ultrasound reception only is executed.

18. The ultrasound image processing apparatus according to claim 13, wherein said plurality of first image data are collected based on an operation timing of a freeze instruction to freeze display of the first image data.

19. The ultrasound image processing apparatus according to claim 13, wherein said plurality of first image data are collected with timing for injecting a contrast medium into a subject used as a reference.

20. The ultrasound image processing apparatus according to claim 13, wherein said plurality of first image data are collected with timing of flash transmission used as a reference.

21. The ultrasound image processing apparatus according to claim 13, wherein the image generation unit generates the second image data that corresponds to at least one of said plurality of frames in response to a freeze instruction to freeze display of the first image data.

22. The ultrasound image processing apparatus according to claim 13, wherein the image generation unit generates a plurality of the second image data using the dynamic range, and
the ultrasound image processing apparatus further comprises a reproduction unit which loop-reproduces said plurality of second image data.

23. The ultrasound image processing apparatus according to claim 13, wherein the determination unit determines the upper limit value based on maximum brightness of the second image data.

24. An ultrasound image processing method comprising:
deciding an upper limit value based on brightness that corresponds to an echo signal from a subject of a plurality of first image data that correspond to a plurality of frames; and
generating second image data that corresponds to at least one of said plurality of frames by the use of said plurality of image data and a dynamic range defined by at least the upper limit value.

25. The ultrasound diagnostic apparatus according to claim 1, further comprising:
an adjustment unit which adjusts the determined upper limit value, such that an average value of brightness values of said plurality of image data included in the dynamic range is same as a predetermined value or included in a range set based on the predetermined value.
